(19) United States
(12) Patent Application Publication
(10) Pub. No.: US 2005/0015148 A1
(43) Pub. Date: Jan. 20, 2005

(54) Biocompatible wires and methods of using same to fill bone void

(76) Inventors: Lex P. Jansen, Pleasanton, CA (US); Mukund Patel, San Jose, CA (US)

Correspondence Address: Bingham McCutchen LLP, Three Embarcadero Center, Suite 1800, San Francisco, CA, 94111-4067, US

(21) Appl. No.: 10/623,381

(22) Filed: Jul. 18, 2003

(51) Int. Cl. A61F 2/44

(52) U.S. Cl. 623/17.11

(57) ABSTRACT

Devices, kits, and methods are provided for reducing a bone fracture, e.g., a vertebral compression fracture, is provided. The device comprises a plurality of resilient wires composed of a biocompatible material, such as a biocompatible polymer (e.g., polymethylmethacrylate (PMMA)). The wires can be introduced into the cavity of the bone structure to form a web-like arrangement therein. The web-like arrangement can be stabilized by applying uncured bone cement onto the arrangement to connect the wires at their contacts point. The bone cavity can then be filled with a bone growth enhancing medium.

### FIELD OF THE INVENTION

The invention relates to the treatment of bone structures, such as vertebrae, and in particular, to the stabilization of bone fractures.

### BACKGROUND OF THE INVENTION

Spinal injuries, bone diseases, such as osteoporosis, vertebral hemangiomas, multiple myeloma, necrotic lesions (Kummel's Disease, Avascular Necrosis), and metastatic disease, or other conditions can cause painful collapse of vertebral bodies. Osteoporosis is a systemic, progressive and chronic disease that is usually characterized by low bone mineral density, deterioration of bony architecture, and reduced overall bone strength. Vertebral compression fractures (VCF) are common in patients who suffer from these medical conditions, often resulting in pain, compromises to activities of daily living, and even prolonged disability. For example, FIG. 1 illustrates three vertebrae 10, 12, and 14, each with an anterior side 16, a posterior side 18, and lateral sides 20 (only one shown). Vertebrae 10 and 14 are fully intact, while vertebra 12 has a VCF 22 (i.e., the top 24 and bottom 26 of the vertebra 12 have been displaced towards each other).

On some occasions, VCF's may be repaired by vertebroplasty and other spinal reconstruction means. During a vertebroplasty procedure, a bone cement, such as polymethylmethacrylate (PMMA), or other suitable biocompatible material, is injected percutaneously into the bony architecture under image guidance, navigation, and controls. The hardening (polymerization) of the cement medium and/or the mechanical interlocking of the biocompatible materials within the medium serve to buttress the bony vault of the vertebral body, providing both increased structural integrity and decreased pain associated with micromotion and progressive collapse of the vertebrae.

In another vertebroplasty-type treatment option, referred to by its trademarked name “Kyphoplasty™”, a high-pressure balloon is inserted into the structurally compromised vertebral body, often through a cannula. The balloon is then inflated under high pressure. It is claimed that the expanding balloon disrupts the cancellous bone architecture and physiological matrix circumferentially and directs the attendant bony debris and physiologic matrix toward the inner cortex of the vertebral body vault. The balloon is then deflated and removed, leaving a bony void or cavity. The remaining void or cavity is repaired by filling it with an appropriate biocompatible material, most often PMMA.

Generally, the treatment objectives of vertebroplasty and Kyphoplasty™ are the same—to salvage, reinforce, and restore tissue functions, while mitigating the progressive nature of the indicated diseases. Additionally, in the instance of primary and metastatic tumor indications and treatments, the concentration of biocompatible material or other therapeutic medium within the margins of or proximate to the tumor may improve the therapeutic effect and patient outcome.

Although these interventional procedures are an improvement over previous conservative treatments that consisted of bed rest, pharmaceuticals, and/or cumbersome back braces, these methods still suffer from practical difficulties associated with filling the relevant anatomy with the therapeutic material. For example, both methods fill the entire space available inside the vertebral body with PMMA, not leaving any space for any long-term therapeutic treatment. In addition, heat generated by the exothermic curing reaction of the PMMA causes necroses of the bone tissue anywhere the PMMA interfaces the vertebra. This inhibits the bone tissue from performing any self-healing activities. Also, the PMMA shrinks several percentages during curing, leaving a “ball” of PMMA loose within the vertebra void. As a result, further degradation or collapse of the treated vertebra may occur.

Currently, the majority of the treated patients are in their seventies, have osteoporosis, and have a relatively short (single digit) life expectancy. Treating them with vertebroplasty or Kyphoplasty™ serves them well. There are, however, much younger patients (with decades worth of life expectancy) presenting collapsed vertebrae caused by injuries not related to osteoporosis. For these younger patients it is very important to receive treatment that has long-term benefits, ensuring a quality of life, continued participation in the workforce and a self-sufficient life style.

Consequently, there is a significant need to provide an improved means for treating bone fractures, such as vertebral compression fractures.

### SUMMARY OF THE INVENTION

In accordance with a first aspect of the present inventions, a device for treating a bone structure (e.g., a vertebra) having a cavity is provided. The device comprises one or more elongate resilient wires composed of a biocompatible material, e.g., polymethylmethacrylate (PMMA) or thermoplastic PMMA polymer, such as Acrylic, resin extruded as wires or monofilament. The wire(s) are configured to be introduced in the cavity of the bone structure. If a plurality of wires are provided, they can be introduced within the bone structure to form a web-like arrangement of wires within the cavity. If the bone structure has a compression fracture (e.g., a vertebral compression fracture), the web-like arrangement may be configured to at least partially reduce the compression fracture.

In accordance with a second aspect of the present inventions, a kit for treating a bone structure (e.g., a vertebra) having a cavity is provided. The kit comprises a plurality of biocompatible laterally resilient wires. By way of non-limiting example, the wires can be composed of a polymer, such as PMMA. The kit further comprises a cannula configured for introducing the wires within the cavity of the bone structure in a web-like arrangement.

The kit may optionally comprise device (e.g., a sprayer, syringe, or injector) configured for applying uncured bone cement (e.g. PMMA) onto the web-like arrangement of wires in a controlled manner, so that the wires can be connected together at their points at contact, thereby stabilizing the web-like wire arrangement. The kit may further optionally comprise a plunger assembly configured to be introduced within the cannula to apply a bone growth inducing material between the resilient wires in the web-like arrangement.

In accordance with a third aspect of the present invention, a method of treating a bone structure (e.g., a vertebral body) is provided. The method comprises introducing a plurality of biocompatible wires within the bone structure to create a web-like arrangement within the cavity of the bone structure. By way of non-limiting example, the wires can be composed of cured bone cement, such as PMMA. The method may optionally comprises applying uncured bone cement onto the web-like arrangement (e.g., by spraying) to interconnect the wires together at points of contact. Preferably, the layer of uncured bone cement that comes in contact with the bone tissue is so thin that no or minimal necrosis of the bone tissue occurs. The method may also optionally comprise applying a bone growth inducing material between the wires, thereby inducing bone growth within the bone structure. If the bone structure comprises a fracture (e.g., a vertebral compression fracture), the method may comprise at least partially reducing the compression fracture by forming the web-like arrangement of wires within the cavity of the bone structure.

### BRIEF DESCRIPTION OF THE DRAWINGS

The drawings illustrate the design and utility of preferred embodiment(s) of the invention, in which similar elements are referred to by common reference numerals. In order to better appreciate the advantages and objects of the invention, reference should be made to the accompanying drawings that illustrate the preferred embodiment(s). The drawings, however, depict the embodiment(s) of the invention, and should not be taken as limiting its scope. With this caveat, the embodiment(s) of the invention will be described and explained with additional specificity and detail through the use of the accompanying drawings in which:

FIG. 1 is a lateral view of three vertebra, two of which are normal, and one of which has a compression fracture;

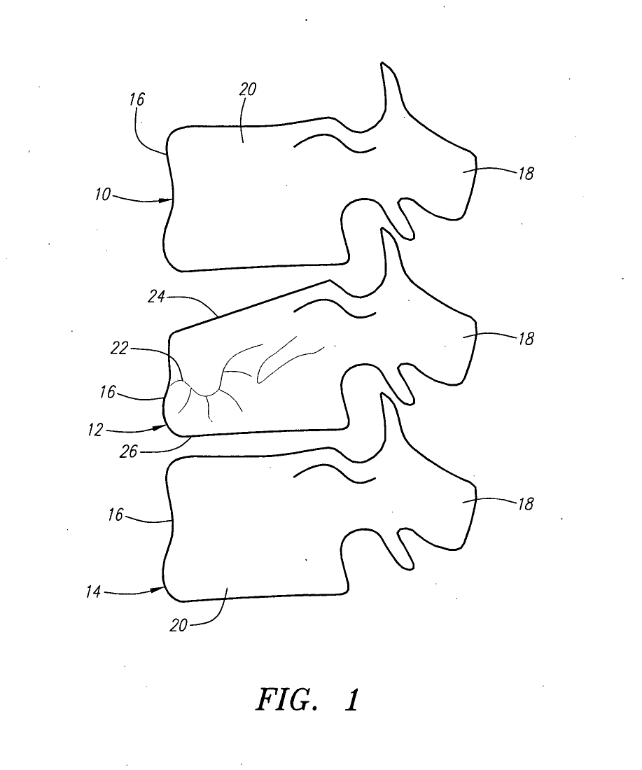

FIG. 2 is a perspective view of a vertebral compression fracture reduction kit constructed in accordance with a preferred embodiment of the present inventions;

FIG. 3 is a partially cut-away top view of a lumbar vertebra;

FIG. 4A is a lateral view of posterior transpedicular access route to the anterior vertebral body shown in FIG. 3;

FIG. 4B is a top view of posterior transpedicular and parapedicular access routes to the anterior vertebral body shown in FIG. 3; and

FIGS. 5-10 are lateral views of a method of using the kit of FIG. 2 to treat a vertebral compression fracture.

### DETAILED DESCRIPTION OF THE PR F RRED EMBODIMENTS

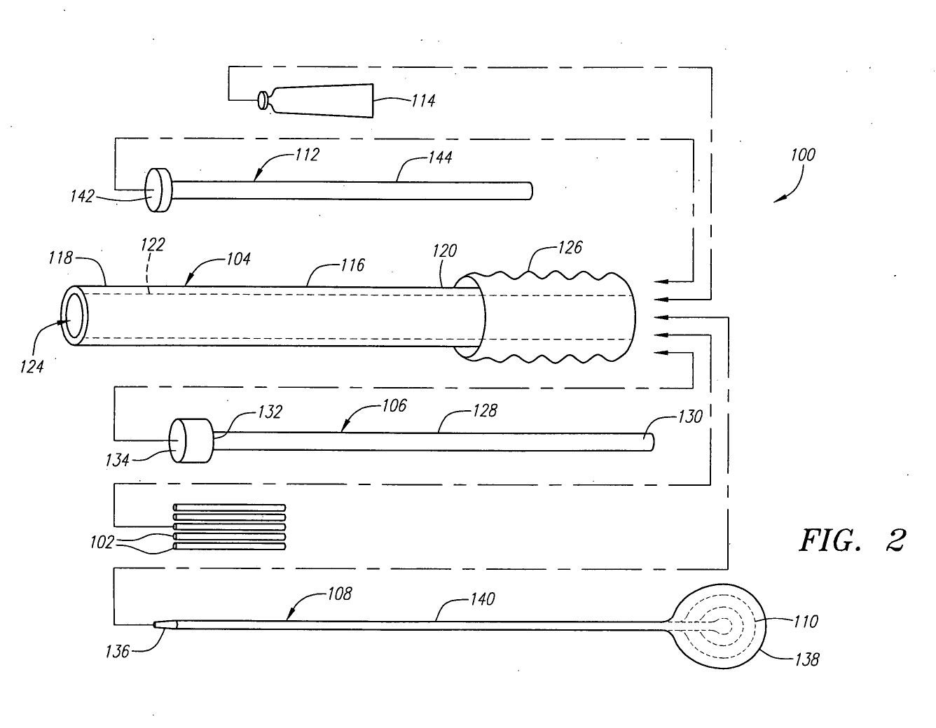

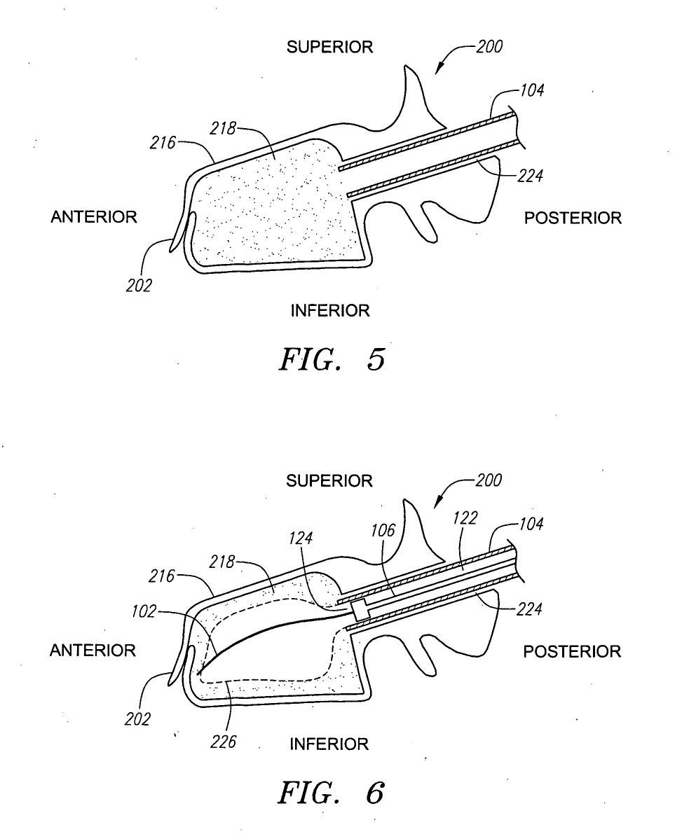

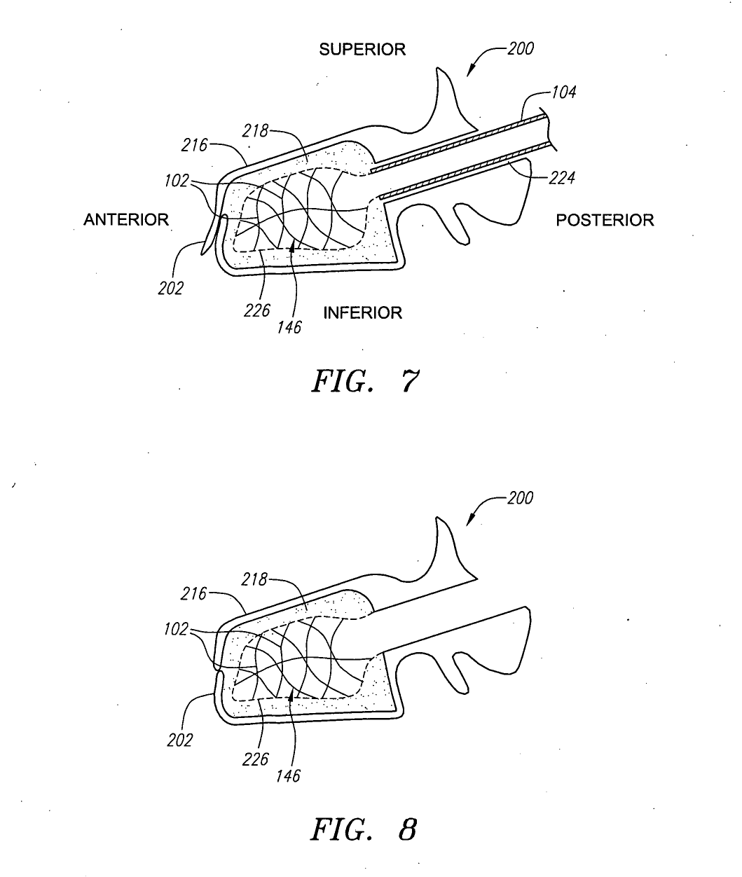

Referring to FIG. 2, a bone fracture treatment kit 100 constructed in accordance with one preferred embodiment of the present inventions is illustrated. The kit 100 can be used for treating a compression bone fracture, and specifically, a compression fracture 202 within a vertebra 200 (shown in FIGS. 4-10). The kit 100 generally comprises a plurality of support wires 102, a delivery member, and specifically a cannula 104, for delivery of therapeutic agents (e.g., the wires 102 and a therapeutic medium) into the vertebra 200, a wire driver 106 for pushing the wires 102 through the cannula 104 into the vertebra 200, an optional spraying device 108 for applying an uncured bone cement 110 to the support wires 102 to stabilize the support wires 102 within the vertebra 200, and an optional plunger assembly 112 for forcing a therapeutic medium 114, and specifically a bone growth inducing medium, through the cannula 104 and into the vertebra 200 between the support wires 102.

Referring still to FIG. 2, the cannula 104 comprises a shaft 116 having a distal end 118 and proximal end 120, a lumen 122 terminating in an exit port 124 at the distal end 118 of the cannula shaft 116, and a handle 126 mounted on the proximal end 120 of the cannula shaft 116. To facilitate introduction into the bone structure vertebra 200, the cannula shaft 116 is preferably stiff (e.g., it can be composed of a stiff material, or reinforced with a coating or a coil to control the amount of flexing), so that the cannula shaft 116 can penetrate the vertebra 200 without being damaged. The materials used in constructing the cannula shaft 116 may comprise any of a wide variety of biocompatible materials. In a preferred embodiment, a radiopaque material, such as metal (e.g., stainless steel, titanium alloys, or cobalt alloys) or a polymer (e.g., ultra high molecular weight polyethylene) may be used, as is well known in the art. Alternatively, if supported by a rigid member during introduction into the vertebra 200, the cannula shaft 116 may be flexible.

The outer diameter of the cannula shaft 116 is preferably less than ½ inch. For transpedicular or extrapedicular approaches, the diameter of the cannula shaft 116 is preferably less than 3/6 inch. A typical cannula size is 11 and 13. Other dimensions for the outer diameter of the cannula shaft 116 may also be appropriate, depending on the particular application or clinical procedure. The cannula lumen 122 should have an inner diameter so as to allow the wires 102 to be delivered within the lumen 122, as will be described in further detail below. In the illustrated embodiment, the profile of the cannula lumen 122 is circular, but can be other shapes as well. In the illustrated embodiment, the distal tip of the cannula shaft 116 is blunt. In this case, the thickness and cross-sectional profile of the cannula shaft 116 is small enough, so that the distal tip can be used as a cutting or deforming tool for boring or coring through bone structure. Alternatively, the distal tip of the cannula shaft 116 may be advantageously sharpened or wedged to facilitate its introduction into the bone structure. Even more alternatively, a stilette (not shown) can be introduced through the cannula lumen 122 to provide an independent means for boring through the bone structure. In this manner, bone cores will not block the cannula lumen 122, which may otherwise prevent, or at least make difficult, subsequent delivery of the wires 102 and other therapeutic materials.

The wire driver 106 comprises a driver shaft 128 having a proximal end 130 and distal end 132, and a driver head 134 formed at the distal end 132 of the shaft 128. The wire driver 106 is sized to slide within the cannula lumen 122 and may be composed of any suitable rigid material, e.g., any of a wide variety of materials, such as plastics, nitinol, titanium, and alloys. In a preferred embodiment, a radiopaque material such as metal (e.g., stainless steel, titanium alloys, or cobalt-chrome alloys) is used. Alternatively, a polymer, such as an ultra high molecular weight polyethylene, may also be used to construct the wire driver 106.

The support wires 102 are configured to be introduced through the cannula lumen 122 into the vertebra 200. The wires 102 are laterally resilient, so that when introduced into the vertebra 200 they engage each other, as well as the inner surface of the vertebra 200, in an interfering relationship to form a web-like arrangement that internally supports the vertebra 200, as will be described in further detail below. The support wires 102 can be composed of any stiff, yet resilient biocompatible material (such as, e.g., cured polymethylmethacrylate (PMMA) cement, thermoplastic PMMA polymer, such as Acrylic resin, polyurethane, acetl, polyester, nylon, ceramic, stainless steel, or nitinol) that has been drawn into the shape of the wires or monofilament 102.

Referring still to FIG. 2, the spraying device 108 comprises a spray head 136, a pump 138 for housing the uncured bone cement 110, and an elongate tube 140 fluidly coupled between the spray head 136 and the pump 138. Preferably, the uncured bone cement 110 exhibits a relatively low viscosity to allow it to be sprayed into a mist. For example, a reformulated PMMA can be used. The spray head 136 and elongate tube 140 are sized to be disposed within the cannula lumen 122. Thus, the spraying device 108 can be operated to provide a spray or mist of the uncured bone cement 110 within the vertebra 200 in order to coat and facilitate stabilization of the web-like arrangement of support wires 102.

The plunger assembly 112 includes a plunger head 142, which is configured to be slidably received into the cannula lumen 122, and a plunger shaft 144 on which the plunger head 142 is mounted. The plunger shaft 144 can be disposed within the cannula lumen 122, allowing for the user to longitudinally displace the plunger head 142 within the cannula lumen 122. The proximal end of the plunger shaft 144 may be coupled to any appropriate controller means to aid in proximal displacing the plunger head 142. Alternatively, the plunger head 142 may be manually displaced.

The plunger shaft 144 is preferably flexible, allowing it to conform to any curves in the cannula shaft 116 without breaking. It may be composed of the same materials as the cannula shaft 116. Alternatively, the plunger shaft 144 may be made from a cable or braided material composed of a suitable material, such as titanium. Ultimately, the type of material selected for the plunger shaft 144 will depend on the viscosity of the bone growth enhancing medium 114 to be implanted within the vertebra 200. For example, a highly viscous material may require a plunger shaft 144 with a high tensile strength, such as braided titanium.

The bone growth enhancing medium 114 may include any one of several natural or artificial osteoconductive, osteoinductive, osteogenic or other fusion enhancing materials. Some examples of such materials are bone harvested from the patient, or bone growth inducing material such as, but not limited to, hydroxyapatite, hydroxyapatite tricalcium phosphate, or bone morphogenic protein.

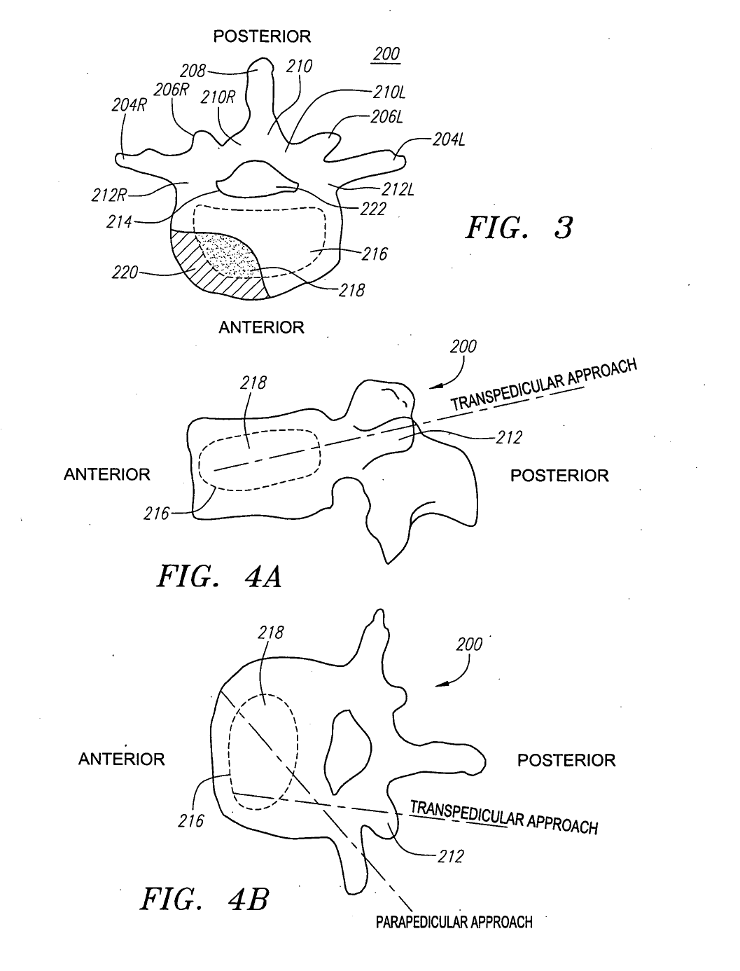

Although, as noted above, use of the bone fracture treatment kit 100 is not limited to treatment of vertebral ailments, such procedures are discussed here for exemplary purposes. Before discussing such methods of operation, various portions of the vertebra are briefly discussed. Referring to FIG. 3, the posterior of the vertebra 200 includes right and left transverse processes 204R, 204L, right and left superior articular processes 206R, 206L, and a spinous process 208. The vertebra 200 further includes a centrally located lamina 210 with right and left lamina 210R, 210L, that lie in between the spinous process 208 and the superior articular processes 206R, 206L. Right and left pedicles 212R, 212L are positioned anterior to the right and left transverse processes 204R, 204L, respectively. A vertebral arch 214 extends between the pedicles 212 and through the lamina 210. The anterior of the vertebra 200 includes a vertebral body 216, which joins the vertebral arch 214 at the pedicles 212. The vertebral body 216 includes an interior volume of reticulated, cancellous bone 218 enclosed by a compact cortical bone 220 around the exterior. The vertebral arch 214 and vertebral body 216 make up the spinal canal, i.e., the vertebral foramen 222, which is the opening through which the spinal cord and epidural veins pass.

Referring now to FIGS. 4-10, a method of using the kit 100 to treat a compression fracture 202 within a vertebra 200 will now be described. First, the patient is preferably placed in a supine position in order to relieve the pressure on the vertebra 200. Then, the physician inserts the cannula 104 into the vertebral body 216 using any one of a variety of approaches. For example, as depicted in FIG. 4A, in a transpedicular approach, access to the cancellous bone 218 in the vertebral body 216 is gained through the pedicles 212. Alternatively, as depicted in FIG. 4B, a parapedicular approach may be used in which access is gained through the side of the vertebral body 216 beside the pedicles 212. This approach may be selected if the compression fracture 202 has resulted in the collapse of the vertebral body 216 below the plane of the pedicles 212. Still other physicians may opt for an intercostals approach through the ribs (not shown) or a more clinically challenging anterior approach (not shown) to the vertebral body 216.

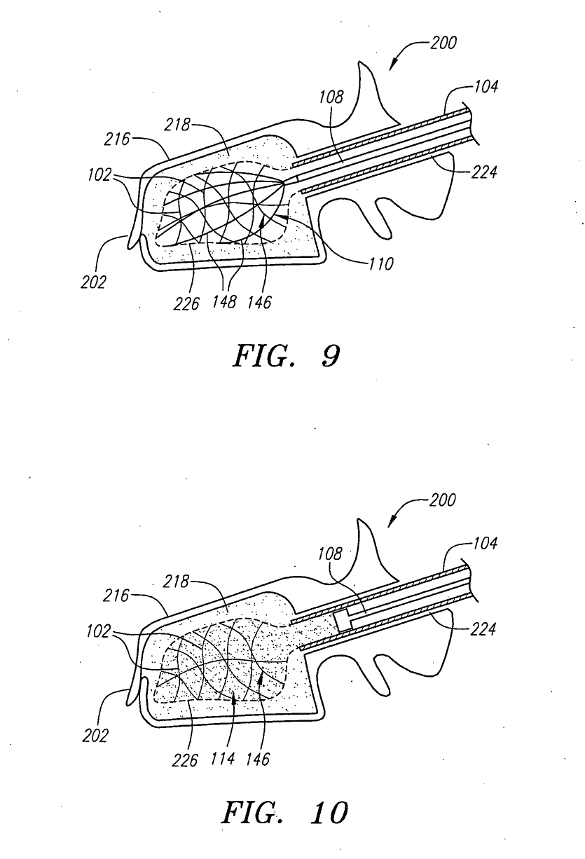

In any event, access to the interior of the vertebral body 216 can be gained by using the cannula 104 to bore into the vertebra 200, thereby creating a channel or passage 224 that houses the cannula 104, as illustrated in FIG. 5. Torsional and/or axial motion may be applied to the cannula 104 to facilitate boring of the vertebra 200. The torsional and/or axial motion may be applied manually or mechanically (i.e., by a machine). An object, such as a hammer or a plunger, may also be used to tap against the handle 126 (shown in FIG. 2) of the cannula 104 in order to facilitate boring into the vertebra 200. Alternatively, a stilette (not shown) that can be introduced through the cannula lumen 122 can be used to create the passage 224, or a separate drill can be used to bore the passage 224 prior to placement of the cannula 104. Even more alternatively, the cannula 104 can be introduced into the interior of the vertebral body 216 through a naturally occurring bore or passage in the vertebra 200 formed as a result of the compression fracture 202.

Once the cannula 104 has been properly placed, a support wire 102 is introduced into the cannula lumen 122, the wire driver 106 is inserted into the cannula lumen 122 and engaged with the support wire 102, and the driver 106 is then distally pushed through the cannula lumen 122 to convey the support wire 102 through the cannula lumen 122, and out the exit port 124 into the cancellous bone 218 of the vertebral body 216, as illustrated in FIG. 6.

The wire driver 106 is then removed from the cannula lumen 122, and the process is then repeated using additional support wires 102 until a suitable web-like arrangement 146 is constructed, as illustrated in FIG. 7. Due to the resiliency of the web-like arrangement 146, a constant force is applied to the superior and inferior sides of the vertebra 200, so that not only is degradation and shrinkage of the vertebra 200 eliminated, the height restoration of the anterior section of the vertebral body 216 will eventually be increased, as illustrated in FIG. 8. Optionally, prior to insertion of the support wires 102, a separate fracture reduction device can be inserted into the vertebral body 216 via the cannula 104 or a separate cannula in order to ensure that the compression fracture 202 is completely reduced. After the separate fracture reduction device has been removed from the vertebral body 216, the superior and inferior sides of the vertebra 200 may temporarily move towards each other again. The subsequently created web-like arrangement 146 of support wires 102 within the vertebral body 216, however, will displace the superior and inferior sides of the vertebral 200 back to their pre-fracture positions.

As a result, this vertebra restoration will improve the life of the patient by correcting his or her posture back to a more original straight position, improving the internal space available for his or her organs and maximizing personal esthetics. Because the wires 102 have already been precured or made of thermoplastic polymer like Acrylic, there will be no exothermic reaction, thereby eliminating necrosis of the bone tissue.

After the web-like wire arrangement 146 has been fully formed, the spraying device 108 is inserted into the cannula lumen 122 and operated to spray a mist of the bone cement 110 onto the wire arrangement 146, as illustrated in FIG. 9. Alternatively, if the bone cement 110 exhibits a relatively high viscosity so that it cannot be sprayed into a mist, the bone cement 110 can be selectively applied to the wire arrangement 146 using other means (such as a syringe or injector) in a manner that minimizes the inadvertent application of the bone cement 110 on the bone tissue. Once cured, the bone cement 110 will connect the wires 102 together at contact points 148, thereby stabilizing and reinforcing the arrangement 146. Notably, any layer of uncured bone cement that is sprayed on the bone tissue is so thin, or otherwise any amount of uncured bone cement that is inadvertently applied to the bone tissue using other means is so minimal, that only an insignificant amount of necrosis will result.

After the bone cement 110 has cured, the bone growth enhancement medium 114, and then the plunger assembly 112, is introduced into the cannula lumen 122. The plunger assembly 112 is then distally displaced within the cannula lumen 122, thereby forcing the therapeutic medium 114 through the cannula lumen 122, out the exit port 124, and into the interior of the vertebral body 216, as illustrated in FIG. 10. The therapeutic medium 114 flows between the wires 102 of the arrangement 146 and hardens, thereby facilitating healing of the compression fracture 202 and providing increased structural integrity for the vertebra 200. Assuming that an out-patient procedure is performed, the relative long time period required for the bone growth enhancing medium 114 to stimulate the required bone growth may be unacceptable. In this case, a fast curing therapeutic medium that does not cause necrosis of the bone tissue can be used, so that the patient can be quickly placed on his or her feet after completion of the procedure.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

1. A device for treating a bone structure having a cavity, comprising:
a single elongate laterally resilient wire composed of a biocompatible material, the wire configured to be introduced into the cavity of the bone structure.

2. The device of claim 1, wherein the bone structure is a vertebral body.

3. The device of claim 1, wherein the biocompatible material is polymethylmethacrylate (PMMA).

4. The device of claim 1, further comprising a plurality of individual elongate laterally resilient wires, each of which is composed of a biocompatible material and is configured to be introduced into the cavity of the bone structure.

5. The device of claim 4, wherein the bone structure comprises a compression fracture, and wherein the web-like arrangement is configured to at least partially reduce the compression fracture.

6. The device of claim 5, wherein the bone structure is a vertebral cavity and the compression fracture is a vertebral compression fracture.

7. A kit for treating a bone structure having a cavity, comprising:
a plurality of biocompatible laterally resilient wires; and
a cannula configured for introducing the wires within the cavity of the bone structure in a web-like arrangement.

8. The kit of claim 7, wherein the bone structure is a vertebral body.

9. The kit of claim 7, wherein the wires are composed of a polymer.

10. The kit of claim 9, wherein the polymer is polymethylmethacrylate (PMMA).

11. The kit of claim 7, further comprising a device configured for applying uncured bone cement onto the web-like arrangement of wires.

12. The kit of claim 11, wherein the device is configured to be introduced within the cannula.

13. The kit of claim 11, further comprising the uncured bone cement.

14. The kit of claim 13, wherein both the wires and uncured bone cement are composed of polymethylmethacrylate (PMMA).

15. The kit of claim 7, further comprising a plunger assembly configured to be introduced within the cannula to apply a bone growth inducing material between the resilient wires in the web-like arrangement.

16. The kit of claim 15, further comprising the bone growth inducing material.

17. The kit of claim 7, wherein the bone structure comprises a compression fracture, and wherein the web-like arrangement comprises a structure that at least partially reduces the compression fracture.

18. The kit of claim 17, wherein the bone structure is a vertebral cavity and the compression fracture is a vertebral compression fracture.

19. The kit of claim 17, further comprising a separate compression fracture reducing device configured to facilitate reduction of the compression fracture.

20. A method of treating a bone structure, comprising:
introducing a plurality of biocompatible wires within the bone structure to create a web-like arrangement within the cavity of the bone structure.

21. The method of claim 20, wherein the bone structure is a vertebral body.

22. The method of claim 20, wherein the wires are composed of a polymer.

23. The method of claim 20, wherein the wires are composed of polymethylmethacrylate (PMMA).

24. The method of claim 20, wherein the web-like arrangement comprises points of contact between the wires, the method further comprising applying uncured bone cement onto the web-like arrangement of wires to interconnect the wires at the points of contact.

25. The method of claim 24, wherein the uncured bone cement is sprayed onto the web-like arrangement.

26. The method of claim 25, wherein both the wires and uncured bone cement are composed of polymethylmethacrylate (PMMA).

27. The method of claim 20, further comprising applying a bone growth inducing material between the wires.

28. The method of claim 20, wherein the bone structure comprises a compression fracture, the method further comprising at least partially reducing the compression fracture by forming the web-like arrangement of wires within the cavity of the bone structure.

29. The method of claim 28, wherein the bone structure is a vertebral cavity and the compression fracture is a vertebral compression fracture.

30. The method of claim 28, further comprising inserting a separate compression fracture reducing device into the cavity of the bone structure, reducing the compression fracture with the fracture reducing device, and removing the fracture reducing device to relax the compression fracture, wherein the web-like arrangement of wires is formed within the cavity of the bone structure subsequent to the relaxation of the compression fracture.
